(19) United States
(12) Patent Application Publication
(10) Pub. No.: US 2007/0253920 A1
(43) Pub. Date: Nov. 1, 2007

(54) Array of swim care articles and method for providing same to shopper and retailer

(76) Inventors: Keegan G. K. Coulter, Appleton, WI (US); David R. Otts, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/413,474

(22) Filed: Apr. 28, 2006

(51) Int. Cl. A61K 8/19; G06Q 10/00

(52) U.S. Cl. 424/59; 705/1

(57) ABSTRACT

An array of swim care articles includes a disposable swim pant and a sunscreen preparation. A method of promoting the sale of the array of swim care articles to a shopper includes combining the articles into in a common container, wherein the disposable swim pant and the sunscreen preparation are manufactured by or for the same entity. The container of swim care articles is sent to a retailer and positioned in close proximity for individual sale to a shopper. A kit including the array of swim care articles bearing the same trademark may also be provided.

### BACKGROUND OF THE INVENTION

When spending time at a pool or lake with a baby or toddler who may not be completely potty trained, it may be desirable or even required that the child wear a disposable swim pant. It may also be very desirable to use sunscreen to protect the child's delicate skin from harmful UV rays. Other swim care items such as towels, water friendly footwear, hats, sunglasses, UV sensors, after sun lotion, and the like may also be things that a parent or caretaker takes along for the child's swim occasion. However, getting all swim care items together for a vacation or for the first time in a swim season can take some effort, especially if a trip to the store is necessary to obtain items for a child that has outgrown, worn-out, or used up last season's swim care items.

There are many persons that shop for swim care items solely in large retail stores. It is typical for many such retail stores to sell disposable absorbent articles such as swim pants near diapers and training pants. For example, some retailers stock swim pants, diapers and training pants in the children's clothing area of the store. Other large retailers might stock such items adjacent disposable articles used for incontinence or menstruation. However, either area of the store is not typically very near to where the sunscreen is located, and typically not very near to where other seasonal swim care items such as hats, shoes, sunglasses are sold. Therefore, it is not convenient for shoppers to find all the swim care items they may need.

In addition to not being convenient to shoppers, retailers also may find it difficult to sell certain items if they are not prominently placed in the store. However, there is only so much space for prominent placement of items in a store—necessarily; some items are on the highest or lowest shelves where they are not as easily seen by the shopper. A shopper that gets distracted (especially by a toddler shopping companion) may not readily see the swim care items he or she needs, so items may be forgotten. The shopper may later end up at a convenience store or hotel gift shop purchasing sunscreen or swim pants at relatively high prices, and the large retailer may have to sell the forgotten items at clearance prices at the end of a swim season.

Large retailers have been known to sell some seasonal items in the same area of the store. However, this requires much coordination of the retailer warehouse and stocking personnel. Further, it is not necessarily obvious by the shopper that this is being done. While some swim care items may be collocated, it is likely that not all of them are collocated because some seasonal items, if placed in one location, take up large amounts of shelf space, e.g. sunscreen.

As such, there remains a need to provide an array of swim care items to a shopper such that shopping is made easier and the shopper is less likely to forget needed items. A method is further needed to make stocking swim care items easier for the retailer.

### SUMMARY OF THE INVENTION

The invention described herein solves these problems when providing an array of swim care items to a shopper. For example, in one aspect of the invention, an array of swim care products includes a disposable swim pant and a sunscreen preparation; the swim pant and the sunscreen preparation are labeled with a first trademark.

In another aspect of the invention, there is a method of promoting the sale of an array of swim care articles to a shopper. The method includes the step of combining into a common container swim care articles such as a disposable swim pant and a sunscreen preparation. The method further includes the step of sending the container containing the swim care articles to a retailer. The retailer may position the disposable swim pant and the sunscreen preparation in close proximity for individual sale to a shopper.

In yet another aspect of the invention is a kit for promoting an array of swim care products. The kit includes a package system that associates the disposable swim pant and the sunscreen preparation for joint sale to a shopper. Both the disposable swim pant and the sunscreen preparation bear the same trademark.

Numerous features and advantages of the present invention will appear from the following description. In the description, reference is made to the accompanying drawings which illustrate various embodiments of the invention. Such embodiments do not represent the full scope of the invention. Reference should, therefore, be made to the claims herein for interpreting the full scope of the invention.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 is a perspective view of one embodiment of an array of the present invention, showing a swim pant and a sunscreen preparation packaged in a bottle;

FIG. 1A is a side perspective view of the swim pant of FIG. 1;

FIG. 2 is a package of the swim pants of FIG. 1;

FIG. 3 is a perspective view of an embodiment of an application device for applying a sunscreen preparation, the application device in the form of a mitt;

FIG. 4 is a bottom elevation of the application device of FIG. 3, taken at lines 4-4;

FIG. 5 is a front perspective view of another embodiment of an application device for applying a sunscreen preparation, the application device in the form of a wipe;

FIG. 6 is a perspective view of one embodiment of a common container for the array of the present invention, in the form of a point-of-purchase display;

FIG. 7 is a perspective view of another embodiment of a common container for the array of the present invention, in the form of a point-of-purchase display;

FIG. 8 is a perspective view of a common container for the array of the present invention, taking the form of a shipping container holding the point-of-purchase display of FIG. 8;

FIG. 9 is a schematic diagram showing how a field of vision is calculated; and

FIG. 10 is an embodiment of a kit of the array of the present invention.

Repeat use of reference characters in the present specification and drawings is intended to represent the same or analogous features or elements of the present invention.

### DETAILED DESCRIPTION OF THE INVENTION

It is to be understood by one of ordinary skill in the art that the present discussion is a description of exemplary embodiments only, and is not intended as limiting the broader aspects of the present invention.

The present disclosure is generally directed to an array of swim care articles including at least a swim pant and a sunscreen preparation. As the term “swim care” implies, these items can be used to care for oneself or others while participating in water-related activities at a pool, beach, backyard, or the like. For instance, each article may be used to help keep a person safe and/or clean at swimming lessons, at a beach, or just washing a car. Swim pants, sunscreen and other forms of swim care articles are discussed herein as they relate to a method of promoting the sale of such articles.

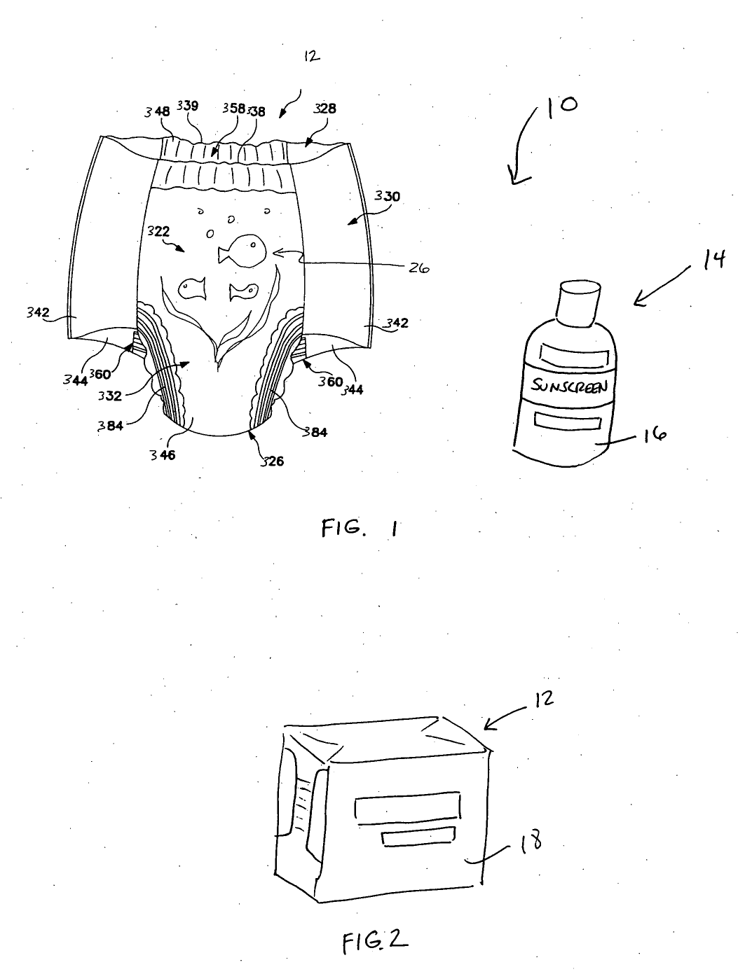

As seen in FIG. 1, an array 10 in a most basic form includes a disposable swim pant 12 and a sunscreen preparation 14. As described herein, the swim pant 12 may be sold individually or in multiples. Likewise, the sunscreen preparation 14 may be sold in packages containing an amount for a single application or multiple applications. In addition, the sunscreen preparation 14 may be contained in bottles, flexible packets and the like, or pre-applied to an application device. The array 10 may include one or more of the swim care articles listed above, and may be shipped and/or sold from a container such as a point-of-purchase display. The various aspects of the invention are described in the following.

Swim pants are used by children who have not completed day-time potty training. In general, a swim pant 12 is very much like a training pant, except it contains little or no superabsorbent materials. “Superabsorbent” or “superabsorbent material” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. By omitting superabsorbent materials from the swim pant 12, it will not swell with water while partaking in water-sports or other wet activities. Therefore, it will not tend to swell and burst and/or slide down the child's body or droop like a diaper or training pant.

In the swim pant 12 of FIGS. l and 1A, the front and back regions 322 and 324 together define a three-dimensional pant configuration having a waist opening 358 and a pair of leg openings 360. The waist edges 338 and 339 of the absorbent chassis 332 are configured to encircle the waist of the wearer when worn and provide the waist opening 358 which defines a waist perimeter dimension. Portions of the transversely opposed side edges in the crotch region 326 generally define the leg openings 360. The front region 322 includes the portion of the swim pant 12 which, when worn, is positioned on the front of the wearer while the back region 324 includes the portion of the swim pant 12 which, when worn, is positioned on the back of the wearer. The crotch region 326 of the swim pant 12 includes the portion of the swim pant 12 which, when worn, is positioned between the legs of the wearer and covers the lower torso of the wearer.

The absorbent chassis 332 is configured to contain and/or absorb any body exudates discharged from the wearer. For example, the absorbent chassis 32 may include a pair of elasticized containment flaps 362 which are configured to provide a barrier to the transverse flow of body exudates. More particularly, in terms of swimwear, the containment flaps 362 help prevent the escape of bowel movements from the swim pant 12. Furthermore, the containment flaps 362 provide pre-swim urine leakage protection when the absorbent assembly 350 can no longer acquire the incoming fluid at the rate at which it is being delivered.

The elasticized containment flaps 362 define an unattached edge 364 which assumes an upright, generally perpendicular configuration in at least the crotch region 326 of the swim pant 12 to form a seal against the wearer's body. Suitable constructions and arrangements for the containment flaps 362 are generally well known to those skilled in the art and are described in U.S. Pat. No. 4,704,116, which is incorporated herein by reference.

Absorbent swimwear is designed for leakage prevention prior to swimming and for fecal containment during swimming. When a wearer wears absorbent swimwear into a pool or lake, the swimwear has a tendency to fill up with water. The swim water that enters the swimwear mixes with the bodily excretions contained within the garment and may thereafter exit the garment, carrying with it various microorganisms from the bodily excretions. Solid waste is kept inside the swim pant 12 regardless of the release of the swim water, because the body side liner material 348 is constructed as in a normal absorbent garment, such as a diaper or training pant, to keep bowel movements contained therein.

Swim pant 12 may include graphics on the outer surface of front and back regions 322 and 324, for example graphics 26. It may be desirable for the graphics 26 to have a water-related theme. More details concerning swim pant 12 construction may be found in commonly owned U.S. Pat. Nos. 6,596,920; 6,627,788, 6,822,136; and 6,797,856, incorporated herein to the extent that they are consistent with the present invention. Swim pants may further include an outer shell such as a boxer or skirt. See also, commonly owned US patents and patent applications showing disposable absorbent garments that include some type of outer shell: US2005/0131377 to Franke et al.; U.S. Pat. No. 6,168,585 to Cesco-Cancian; U.S. Pat. Nos. 6,115,847 and 6,009,558 and 5,876,394 to Rosch et al.; U.S. Pat. Nos. 6,192,521 and 5,915,536 to Alberts et al.; EP 1329166 and EP 0955975 to Rosch et al.; U.S. Ser. No. 10/737101 (US20050131382); U.S. Ser. No. 10/735978 (US20050131377); U.S. Ser. No. 10/736069 (US20050131381); U.S. Ser. No. 10/736443 (US20050125879). Each of these references are commonly owned by the assignee of the present invention, and are incorporated by reference to the extent they are consistent with the present invention.

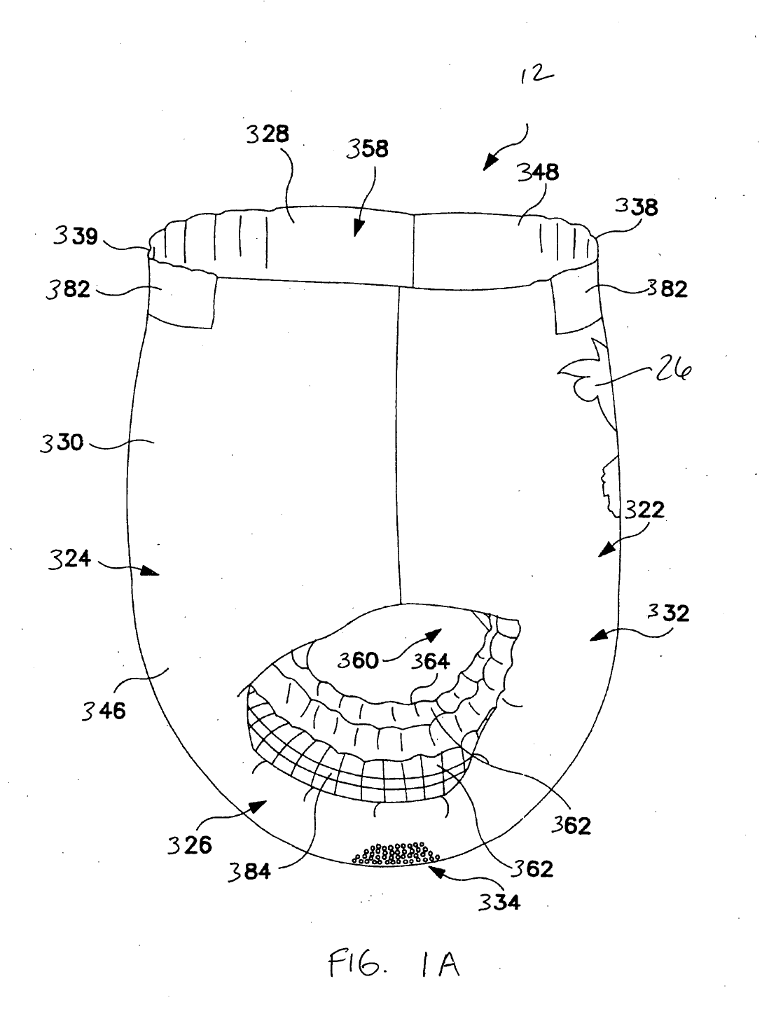

In various aspects of the present invention, the swim pant 12 may be disposed in a package, either individually or by multiples. In various aspects, the package may include a rigid material such as cardboard, molded plastic, and the like. In other aspects, the package may be a flexible consumer package 18 such as that shown in FIG. 2. As used herein, the term “flexible consumer package” refers to non-rigid containers, such as polyethylene bags, that are adapted to contain swim pants and are adapted to be presented to a consumer. Package 18 contains a plurality of swim pants 12. Swim pants 12 may be further individually wrapped and prior to being packaged in package 18 (not shown).

Sunscreen liquid preparations can take the form of a lotion, lotion spray, mist spray, gel, roll-on applicator products, or solid stick-form products. The lotion spray can either be continuous as delivered by pressurized packaging or discontinuous, e.g. delivered through a mister or trigger-based applicator. Liquid formulations for the lotion spray and roll-on products have sufficient lower viscosity relative to a standard lotion to enable delivery through the application device. Gels may contain the appropriate viscosity modifiers and structurants to ensure appropriate gel formation for product integrity and easy delivery and spreading on the skin. The stick product may contain aliphatic hydrocarbons and waxes to ensure its maintenance as a solid phase at use temperature while enabling melting and transfer to the skin during application. All of these formulations may have one or multiple sunscreen actives as defined herein.

Sunscreen preparation 14 may be formulated from any compound that has at least an SPF 2. For example, the formulation may contain a carrier solvent, one or more ultra-violet light absorbers, an emollient, and a waterproofing agent. Carrier solvents may include water, mineral, or alcohol. UV light absorbers may include one or more of the following: octinoxate, octisalate, avobenzone, homosalate, titanium dioxide, zinc oxide, octacrylene, or the like. The waterproofing agent may include one or more of the following: cetyl, phosphate, acrylates/C12-22 alkyl methacrylate copolymer, or VP/eicosene copolymer. Other optional ingredients include: insect repellant, aloe, botanical extracts, glitter, vitamins, fragrance, or an aesthetic feel enhancer. Regardless of the specific formulation, the sunscreen preparation 14 is a liquid that is packaged for sale to a consumer as described herein.

In one aspect of the invention, the sunscreen preparation 14 is a liquid that is disposed in a container such as a bottle 16. Desirably, bottle 16 is a breakage-resistant material such as plastic, as is well known in the art of packaging. For example, bottle 16 may be constructed from a polyethylene plastic or the like. In other aspects, the container may be a flexible consumer package (not shown) similar to how individual condiments are packaged. As used herein, the term “flexible consumer package” refers to non-rigid bags, such as polyethylene packets, that are adapted to a liquid sunscreen preparation and are adapted to be presented to a consumer. The material from which the sunscreen preparation container is fabricated is not critical to the present invention.

In various aspects of the invention, the sunscreen preparation 14 is presented to a consumer in varied amounts per container. For example, there may be a quantity of sunscreen preparation 14 in a container to last for several applications. Such a container may last a swimmer for an entire swim season. In one aspect of the invention the container contains less than about 80 ml of sunscreen preparation. In another aspect of the invention the container contains less than about 60 ml of sunscreen preparation. In yet another aspect of the invention the container contains less than about 40 ml of sunscreen preparation.

In a further aspect of the invention, the sunscreen preparation is presented to the consumer either pre-applied to or in conjunction with an application device. For instance, a container of sunscreen preparation 14 may be presented to the consumer in combination with an application device in the form of a mitt 22 (FIG. 3) or a disposable wipe 24 (FIG. 5).

Mitt 22 may be made for limited use and then disposed of instead of laundering, or may be made for multiple uses and capable of withstanding laundering between uses. In one aspect, disposable mitt 22 may be constructed from an all natural fiber material, such as cellulosic fibers, cotton linters, rayon, flax, or the like, or of an all synthetic fiber material, such as polypropylene fibers, polyester fibers, and polyethylene fibers. Mitt 22 may also or instead be made from synthetic fibers include water-soluble or water-dispersible polymers, such as polyethyloxazoline, polyethylene oxide, polyvinyl acetate, polyvinyl alcohol, water-dispersible acrylic polymers, starch polymers, or the like.

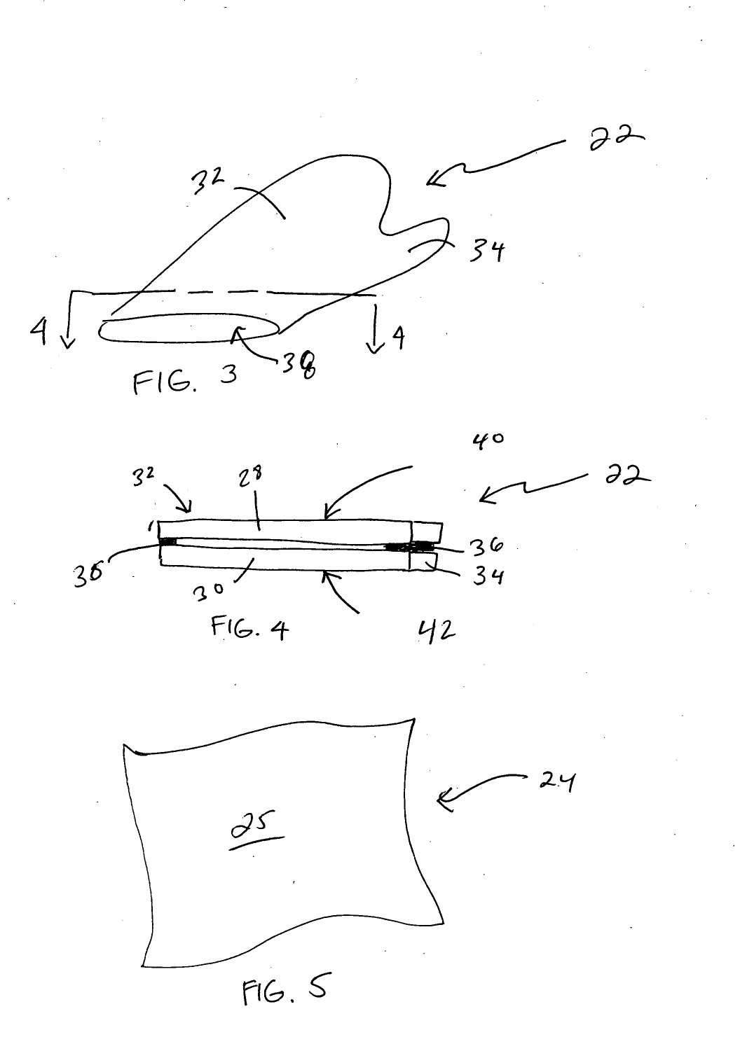

A disposable mitt 22 may have sunscreen preparation 14 pre-applied thereto prior to being packaged for presentation to a consumer, or may be presented to the consumer with a container of sunscreen preparation 14 so that the consumer may apply a desired amount of sunscreen preparation to the mitt just prior to use. For example, a disposable mitt 22 as seen in FIG. 4 is constructed from two sheets of nonwoven material, a first sheet 28 and a second sheet 30. The sheets 28 and 30 are about identical in shape, the shape being similar to a mitten having a palm portion 32 and a thumb portion 34. The sheets are aligned and joined together with a bond 36 around all edges except opening 38 with an adhesive, thermal bond, pressure seal, or other means of connection as is known in the art. Once sheets 28 and 30 are joined, mitt 22 has a first exposed surface 40 and a second exposed surface 42. Sunscreen preparation 14 may be pre-applied to one or both surfaces 40 and 42 prior to being disposed in a container for presentation to a consumer. It is further contemplated that a mitt may be used such as that disclosed in U.S. Pat. No. 5,649,336, incorporated herein to the extent that it is consistent with the present invention.

In another aspect the invention, a launderable mitt 22 may be constructed from a woven fabric, a nonwoven fabric, or any suitable fabric used to make bath towels, wash cloths, and the like. Desirably, the launderable mitt 22 is presented to the consumer with a container of sunscreen 14.

Mitt 22 may be decorated with graphics (not shown) that correspond to an overall theme or the swim pant graphics 26 or sunscreen container graphics (not shown).

Referring now to FIG. 5, in various other aspects of the invention, the sunscreen preparation may be applied to a wipe. A wipe is a sheet substrate that can hold a predetermined amount of liquid. The substrate may be a nonwoven material such as foam, coform, SMS, spunbond, or a woven material such as a cotton fabric or the like. Other substrates maybe used such as that described in commonly owned U.S. Pat. Nos. 6,028,018 to Amundson et al.; 4,720,415 to Vander Weilen et al.; 5,972,361 to Fowler et al.; and 5,385,775 to Wright. Similar to a “wet wipe” used for cleaning the body, sunscreen preparation 14 may be pre-applied to wipe 24, such as at a surface 25, and then packaged for presentation to a consumer. In the alternative, sunscreen preparation 14 may be applied to the wipe by the consumer just prior to use. Like the mitt 22, wipe 24 may be intended to be reused after laundering, or disposable and not readily launderable.

Unlike swim pants 12, sunscreen preparation 14 may be used by children and adults alike. However, to help remind a child to wear sunscreen, it may be desirable to associate with the sunscreen preparation 14 a characteristic that is appealing to children, such as brightly colored packaging, a fruity scented fragrance (or other scents that children enjoy), a sparkling glitter suspended in the liquid and visible on skin after application, a favorite theme or trademark on the packaging, and the like.

In various aspects of the present invention, the swim pant 12 and sunscreen preparation 14 may be branded with the same trademark or with different trademarks from the same trademark owner. An “owner” is defined broadly to include separate divisions or subsidiaries of a parent company or business entity. Thus, if two companies are owned by a common business entity yet own different trademarks, then the trademarks are considered to have common ownership. In one aspect of the invention, the swim pant 12 and sunscreen preparation 14 are associated with a first commonly owned trademark. One example of a trademark owned by the assignee of this invention is “HUGGIES.” However, this particular mark is not critical to the present invention-any desired trademark may be used. For example, the swim pant package 18 and the sunscreen bottle 16 may both be marked with the HUGGIES trademark.

In various other aspects of the present invention, the swim pant 12 and/or the sunscreen preparation are marked with a commonly owned second trademark. As another non-limiting example, the swim pant package 18 and/or the sunscreen bottle 16 may both be marked with the LITTLE SWIMMERS trademark. It is further contemplated that when sunscreen preparation 14 does not bear a second trademark and the swim pant 12 does bear a second trademark, then the sunscreen preparation 14 may bear yet a third trademark.

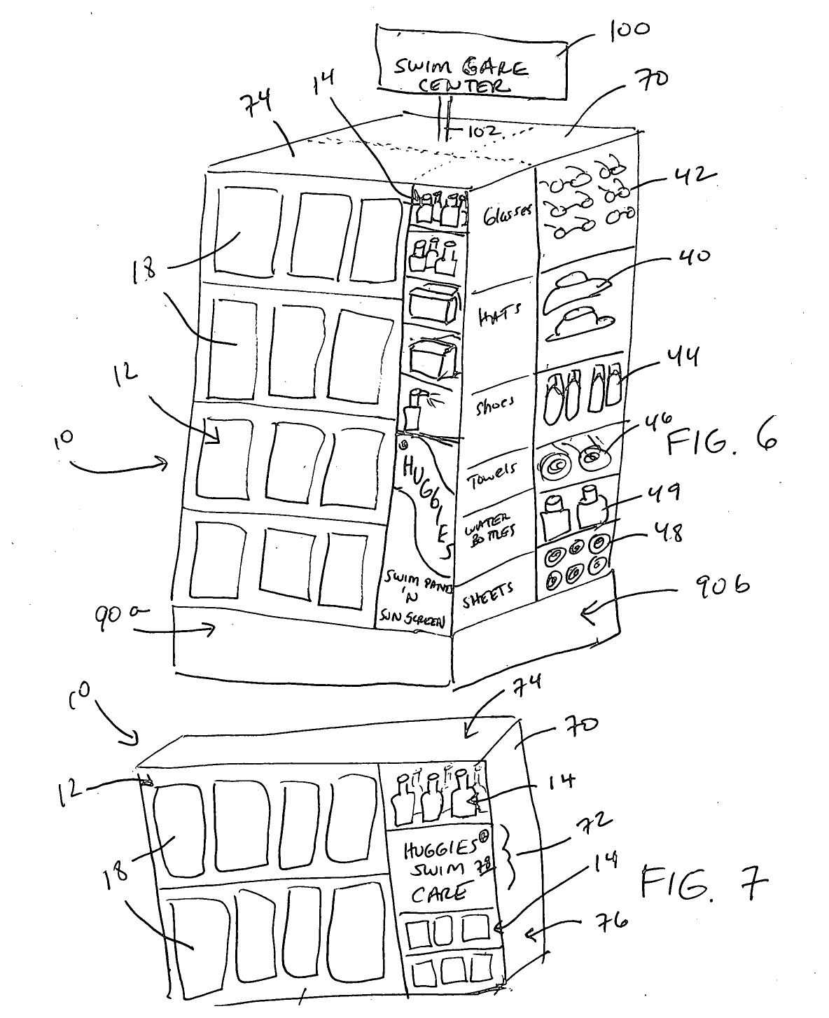

Referring to FIG. 6, array 10 may further include one or more other swim care articles. For example, it is contemplated the other swim care articles might be hats 40, sunglasses 42, swim wear (not shown), UV sensors (not shown), UV protective clothing (not shown), after-sun lotion (not shown), sun protective drugs (not shown), foot wear 44, swim toys (not shown), towels 46, disposable sheets 48, disposable cleansing wipes (not shown) and/or water bottles 49. The hats 40 may be wide-brimmed, baseball-style, or any type of hat that is worn on the head as is known in the art. The sunglasses 42 may be of any shade, size, tint, or style. The purpose of the sunglasses is to at least partially block light rays from entering a wearer's eyes. UV sensors maybe in the form of small stickers that are placed on a sun-exposed portion of skin or clothing to indicate when more sunscreen is needed or when sun-exposure should be ceased. After-sun lotion is a topical lotion for moisturizing the skin. Sun protective drugs may be ingested to counteract or prevent effects of sun exposure on the skin, e.g. in pill or liquid form. Foot wear 44 may include sandals or shoes, especially water-durable shoes adapted to be worn at a pool or beach, e.g. flip-flops sandals or water shoes. Swim toys may be water-proof or water resistant toys (e.g. boats, pails, shovels, beach balls, etc.) or water flotation devices. Towels 46 may be launderable and water absorbent, such a beach towel as is known in art. Disposable sheets 48 may made of a nonwoven fabric that may be launderable a limited number of times, (see commonly owned U.S. patent application Ser. No. 09/865,179, incorporated herein to the extent that it is consistent with the present invention). Water bottles 49 may be breakage resistant bottles, such as polyethylene, polycarbonate, or other plastic, and may be of any configuration as is known in the art. Desirably, the water bottles 49 may be sealable so that they do not leak liquid. Further, water bottles 49 may include a fan and spray mechanism for providing a cooling mist.

In various aspects of the present invention, one or more of the other swim care articles is associated with the first trademark and/or the second trademark. In the alternative, one or more of the other swim care articles may bear a third trademark, and not the first and/or second trademark. It is further contemplated that the one or more of the other swim care articles are manufactured by or for the same business entity. For example, the disposable sheet may be manufactured by the same business entity, and bear a commonly owned third trademark, e.g. NEAT SHEET.

The present invention further includes a method of promoting the sale of array 10 to a shopper. In one aspect of the invention, the method is intended to make it easier for the retailer to position swim care articles in the retail setting for sale to a shopper. The various articles making up the array 10 may be sold through any retailer. Various distributors, hospitals, doctors' offices, and other suitable parties may also be involved. A retailer, for example, receives the various components of the array 10, and places them in close proximity to each other on one or more shelves for sale to a shopper or consumer.

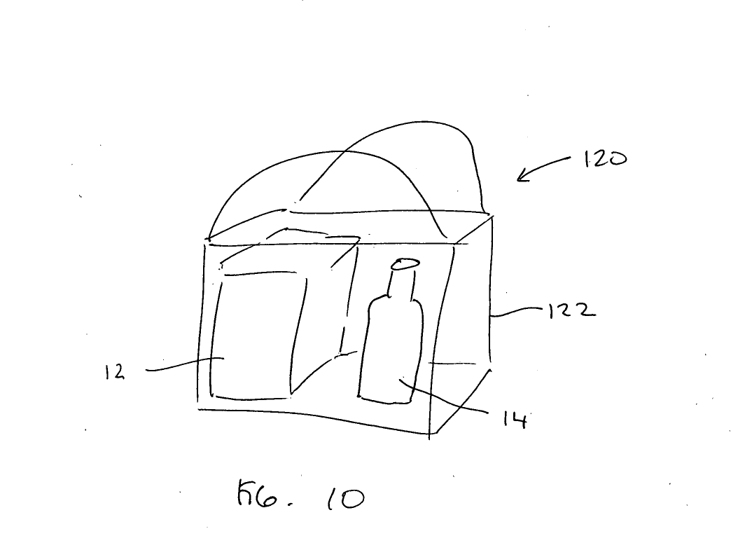

“Close proximity” is defined as (1) item located within a single field of vision of an angle as measured from a focal point or (2) being displayed in a common point-of-purchase display regardless of whether or not they are in a single field of vision. Referring to FIG. 9, the method for determining whether two articles are in a single field of vision is as follows: A blaze orange (or other color that contrasts from the retail environment) pole “P,” approximately 3 meters long and a centimeter in diameter are placed at the outer edge of an article such as a package of swim pants 12 or a container of sunscreen preparation 14. How high the article is located from the floor is not critical. A line “D” is defined between the two poles P1 and P2. Line D is bisected, and a line Dc extends perpendicularly outward from the line D from where it is bisected. A line “L” is defined between a focal point F and the longitudinal axis “X” of each pole, P1 and P2. At the focal point F, an angle “A” is measured between lines “L.” It is contemplated when focal point F is a distance of 2 meters from line D, that the angle A is between about 110 and about 120 degrees. In the alternative, angle A is between about 90 and about 110 degrees. In yet another embodiment, angle A is between about 80 and 90 degrees. In a further embodiment, angle A is less than about 80 degrees.

In one embodiment of the invention, the articles of array 10 are combined into a common container 60, such as a pallet (not shown), crate (not shown), or shipping box as shown in FIG. 8. For example, the common container 60 may contain at least a swim pant 12 and a sunscreen preparation 14 manufactured by or for the same business entity. It is anticipated that the common container may contain 1) a swim pant 12 manufactured (or caused to be manufactured) by the business entity that performed the step of combining the array into container 60, and 2) a sunscreen preparation 14 that is manufactured by a by or for a third party business entity. It is further anticipated that the common container may contain a sunscreen preparation 14 manufactured (or caused to be manufactured) by the business entity that performed the combining of the array into container 60, and a swim pant 12 that is manufactured by a by or for a third party business entity. In addition, the neither the sunscreen preparation 14 nor the swim pant 12 may be made by or for the business entity that performed the step of combining the array into container 60.

The swim pant 12 and the sunscreen preparation 14 may each bear a first trademark as described previously. Desirably, common container 60 contains a plurality of swim pants 12 in a plurality of packages 18. The sunscreen preparation 14 may further be contained in one or more types of containers, such as bottle 18 and/or a flexible packet as described previously. It is further contemplated that the sunscreen preparation 14 may be combined with an application device such as a mitt 22 or wipe 24, whether pre-applied to the application device, or not pre-applied to the application device.

The common container 60 is sent to the retailer so that the retailer may display the array 10 for sale. By combining the swim care articles in a common container 60, the retailer may position the swim pant 12 and the sunscreen preparation 14 in close proximity (as defined above) for individual sale to a shopper.

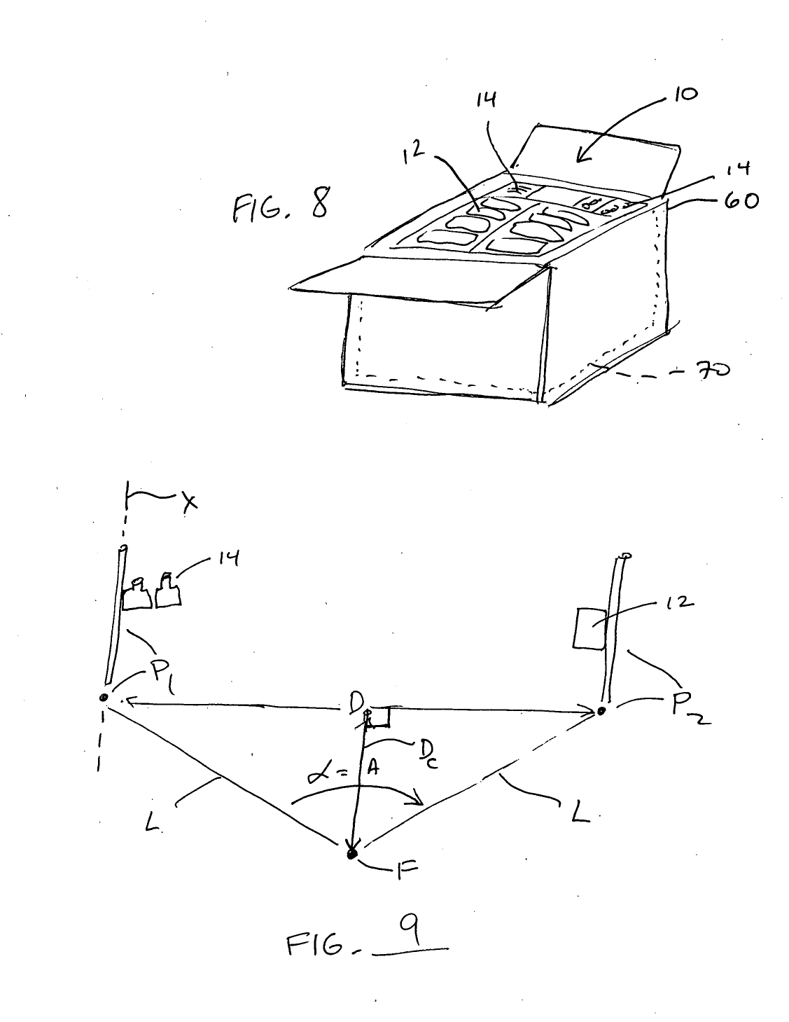

In various aspects of the invention, the common container 60 may either double as or further include a point-of-purchase display unit 70 (“PPD unit 70”) as is known in the art. It is contemplated that a PPD unit may be a display that requires no or very limited assembly by the retailer. For example, a PPD may be filled with product, and need only legs or a stand portion attached thereto by the retailer. Referring to FIGS. 7 and 8, one example of a PPD unit 70 is packed with swim pants 12 and sunscreen preparation 14, and disposed in a common container 60 (e.g. a corrugated box) for shipping and/or handling. The exact type of common container used to contain a PPD unit 70 is not critical to the present invention. Desirably, the common container 60 may be opened by a retailer to reveal the PPD unit 70 filled with swim care articles. The PPD unit 70 may then be removed from common container 60 and displayed in a retail setting such as a store shelf.

Desirably, indicia 72, such as a brand name; information regarding identification, selection, and/or use of the swim care articles displayed in the PPD unit 70; theme related graphics (such as a common character or story-related graphics) and/or coordinated designs may be prominently displayed on an outer surface of PPD unit 70. For instance, the PPD 70 shown in FIG. 7 has a top surface 74, opposite side surfaces 76 (one not shown), and a bottom surface and back surface (also not shown). The particular PPD 70 example of FIG. 7 further includes an optional front panel 78 at a front display surface 77. (It should be noted that a display surface may be a plane) Since the bottom surface of PPD 70 is not seen by shoppers, it may not contain any indicia 72. However, one or more of the other surfaces of PPD 70 may contain indicia. As shown by way of example, the front panel 78 contains indicia 72.

Referring to FIG. 6, in another aspect of the present invention, a PPD unit 70 may be adapted to display at least one additional swim care article, such as hats, sunglasses, swim wear, foot wear, swim toys, towels, disposable sheets, and water bottles, etc. as described herein. Though there are a myriad of configurations that a PPD 70 might have for displaying array 10, it is contemplated that in one embodiment, swim pants 12 and sunscreen 14 may be contained in a PPD unit 70 such they are in a single field of vision as defined herein. For example, if PPD unit 70 is a polyhedron with a top surface 74, a bottom surface (not shown) and at least three display surfaces collectively referred to a surface 90, the swim pants 12 and the sunscreen preparation 14 may be accessible at a single display surface, such as surface 90a. One or more of the additional swim care articles listed above may be accessible from another single display surface 90b. One or more other display surfaces 90 may be configured to (1) not be viewed by shoppers, such as by placing the display surface against a wall, (2) contain indicia or made otherwise suitable to be viewed by shoppers (e.g. a solid color), or (3) have additional swim care articles accessible therefrom. In another aspect of the invention, a swim pant 12 and a sunscreen preparation 14 are displayed on a PPD 70 so that they are not in a single field of vision.

It is further contemplated that there may be a sign or banner 100 disposed on top surface 74. The banner 100 may sit directly on surface 74, or be raised from surface 74 by a support member 102 or any other support member. Banner 100 may contain indicia carrying a trademark, such as the first trademark and/or second trademark on the swim pants 12, or any other trademarks of swim care articles sold from PPD 70. Banner 100 may rotate or be otherwise animated (such as by an electronic display) to attract the attention of a shopper.

Information may be conveyed to the shopper and/or consumer in several ways. For instance, consumer education may be further enhanced by providing indicia on the packaging to indicate various different but associated articles of the array 10.

The indicia may be disposed directly on the actual packaging material, or be a separate information piece, such as a coupon. The indicia may include graphic design features, text, branding, or any other suitable indicia. For example, all of the packages may exhibit similar graphic design components and branding to identify their association, yet have different coloring and illustrations to identify their differences. These indicia may help a consumer to quickly discern which products may be needed for a particular purpose. Likewise, display surfaces 90 may have panels large enough to contain indicia as previously described.

In various aspects, the swim pants 12 and/or the sunscreen 14 may include informational items such as instructions in the use of the product and tips for safe swimming or sun exposure. As used herein, the term “informational item” refers to objects that are provided in addition to array articles such as swim pants 12 or sunscreen preparation 14, are adapted to communicate information to the user and/or shopper of an article of array 10, and are associated with individual components of the array 10. Examples of informational items include cards, paper, electronic media, printing on the packaging, or other suitable media capable of storing and conveying information. In yet another example, the, PPD 70 may be adapted to emit audio information continuously or on demand, as is known in the art.

In various aspects, the informational items associated with the array 10 articles may be adapted to appeal to the specific category of user and/or purchaser to which the array 10 is adapted. The informational items may be adapted, for example, by providing information likely to be of interest to a given category of user and/or purchaser.

For example, a swim pant 12 may be adapted for use by a caregiver for water recreation purposes. An informational item may be associated with the swim pant 12 that is adapted to interest caregivers. The informational item may be a card or pamphlet containing information or instructions about children's health and hygiene, such as safe swimming, sleep habits, thumb sucking, teething, skin health, toilet training; questions to ask a child; jokes; and the like, and combinations thereof. The informational item may additionally or alternatively include addresses for web sites available on the Internet. The web sites may contain information related to issues of interest for caregivers and users of other disposable absorbent articles, including wetness-sensing absorbent articles.

In various other aspects of the method of the invention, the retailer may need to position the components of array 10, such as swim pant 12 and the sunscreen preparation 14, in predetermined portions of the PPD 70 or on store shelves in a manner supplied by the supplier of the array 10, for example, a manufacturer of both the swim pants 12 and sunscreen preparation 14. It may be desirable that all products made by the same manufacturer be positioned a single display surface of a PPD 70, such as surface 90a shown in FIG. 6. It may further be desirable to exhibit the first and/or second trademark used on the swim pants 12 at the surface 90a. It is contemplated, however, that any other swim care articles such as foot wear, hats etc. could be manufactured by or for the same entity that owns at least the first trademark associated with the swim pant 12 and/or sunscreen preparation 14. In this case, the entire PPD 70 may display only swim care articles that bear at least the first trademark. It may be further desirable to position the entire array 10 on store shelves, such that they may be viewed from a single field of vision.

Other aspects of the method of the present invention may include the step of packaging the swim pant 12 in a container 18 bearing the first trademark as described above. It is further contemplated that there be a step of packaging the swim pant 12 in a container 18 that bears yet the second trademark as described above. In addition, the method may include a step of packaging the sunscreen preparation 14 in a package bearing the first trademark, for example bottle 16 or an individual use packet (not shown). The method may further include packaging the sunscreen preparation 14 in a package bearing the second trademark as described herein.

In various aspects of the method of the present invention, there may be a step of associating the first trademark or second trademark with at least one of the additional swim care articles described herein ( e.g. hats, sunglasses, swim wear, foot wear, swim toys, towels, disposable sheets, UV sensors, UV protective clothing, after-sun lotion, and water bottles). Such associate may be made by disposing the first and/or second trademark directly on the article, and/ or by disposing the first and/or second trademark on the article packaging or a label. In addition, the association may further be made using indicia on a display surface of PPD 70 containing the array 10 of swim care articles.

Referring now to FIG. 10, the present invention further includes a kit 120 for promoting an array 10 of swim care products. The kit 120 includes a package system 122 that associates the swim pant 12 and the sunscreen preparation 14 for joint sale to a shopper.

The package system 122 may take many forms, including but not limited to: boxes flexible bags; tape; wrap; string; reusable jars or bins; totes such as sacks, beach or book bags, purses, backpacks, and duffel bags. Any of the packaging systems may be made of materials more suitable for a one time use, or for multiple uses such that the shopper or user may reuse the package system for another purpose once the array 10 has been removed therefrom.

A swim pant 12 with a first trademark may be packaged and sold together in the same package with a sunscreen preparation 14 bearing a first trademark. A swim pant 12 with a first and second trademark may be packaged and sold together in the same package with a sunscreen preparation 14 bearing a first trademark. A swim pant 12 with a first and second trademark may be packaged and sold together in the same package with a sunscreen preparation 14 bearing a first and third trademark.

In various aspect of the kit 122, the sunscreen preparation 14 is packaged in at least one container containing less than about 80 ml of sunscreen preparation. In the alternative, the sunscreen preparation 14 is packaged in at least one container containing less than about 60 ml of sunscreen preparation. In yet another alternative, the sunscreen preparation 14 is packaged in at least one container containing less than about 40 ml of sunscreen preparation.

In various other aspects of the kit 122, the sunscreen preparation 14 is contained in flexible packets or bottles as described previously. Sunscreen preparation 14 may further be pre-applied to a mitt 22 or a wipe 24, as described previously.

In another aspect of the invention, one or more swim care articles (including but not limited to hats, sunglasses, swim wear, foot wear, swim toys, UV sensors, UV protective clothing, after-sun lotion, towels, disposable sheets, and water bottles) may be packaged and sold with swim pant 12 and sunscreen preparation 14.

In another aspect of the invention, one or more non-swim care articles may be packaged and sold with swim pant 12 and sunscreen preparation 14. Non-swim care articles are manufactured by or for the same entity, and may include items such as diapers or body lotion. The non-swim care articles may or may not bear a first trademark in common with the array 10, and/or a second trademark in common with array 10.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged either in whole or in part. Furthermore, those of ordinary skill in the art will appreciate that the foregoing description is by way of example only, and is not intended to limit the invention so further described in such appended claims.

What is claimed:

1. An array of swim care products comprising;
a disposable swim pant; and
a sunscreen preparation;
wherein the swim pant and the sunscreen preparation are labeled with a first trademark.

2. The array of claim 1 wherein the sunscreen preparation is adapted to be ingested by a user.

3. The array of claim 1 wherein the sunscreen preparation comprises a liquid and an application device.

4. The array of claim 3 wherein the application device comprises a mitt.

5. The array of claim 3 wherein the application device comprises a wipe.

6. The array of claim 1 wherein the sunscreen preparation comprises a liquid or solid in a container.

7. The array of claim 6 wherein the container contains less than about 80 ml of sunscreen preparation.

8. The array of claim 6 wherein the container contains less than about 60 ml of sunscreen preparation.

9. The array of claim 6 wherein the container contains less than about 40 ml of sunscreen preparation.

10. The array of claim 1 further comprising one or more of the swim care articles selected from the group consisting of: hats, sunglasses, swim wear, foot wear, swim toys, towels, disposable sheets, UV sensors, UV protective clothing, after-sun lotion, and water bottles.

11. The array of claim 10 wherein a one or more of the swim care articles is associated with the first trademark.

12. The array of claim 1 wherein the disposable swim pant is associated with a second trademark.

13. A method of promoting the sale of an array of swim care articles to a shopper, the method comprising:
combining into in a common container swim care articles comprising a disposable swim pant and a sunscreen preparation; and
sending the container containing the swim care articles to a retailer;
wherein by combining the swim care articles in a common container, the retailer may position the disposable swim pant and the sunscreen preparation in close proximity for individual sale to a shopper.

14. A method according to claim 13 wherein the disposable swim pant and the sunscreen preparation are manufactured by or for the same business entity.

15. The method of claim 14 further including the step of packaging the disposable swim pant in a package bearing a first trademark.

16. The method of claim 15 further including the step of packaging the sunscreen preparation in a package bearing the first trademark.

17. The method of claim 16, and the sunscreen preparation is packaged in a package bearing the first trademark.

18. The method of claim 15 wherein the disposable swim pant is packaged in a package further bearing a second trademark.

19. The method of claim 13 wherein the common container comprises a shipping container.

20. The method of claim 13 wherein the common container comprises a point-of-purchase display unit.

21. The method of claim 20 further including the step of positioning the disposable swim pant and the sunscreen preparation in predetermined portions of the point-of-purchase display unit.

22. The method of claim 20 wherein the point-of-purchase display unit is adapted to display at least one additional swim care article selected from the group consisting of: hats, sunglasses, swim wear, foot wear, swim toys, towels, disposable sheets, UV sensors, UV protective clothing, after-sun lotion, and water bottles.

23. The method of claim 22 further including the step of associating the first or a second trademark with at least one additional swim care article selected from the group consisting of: hats, sunglasses, swim wear, foot wear, swim toys, towels, disposable sheets, UV sensors, UV protective clothing, after-sun lotion, and water bottles.

24. The method of claim 13 further including the step of conveying information to a shopper about the swim care articles.

25. The method of claim 13 wherein the step of combining into in a common container swim care articles comprising a disposable swim pant and a sunscreen preparation is performed by the same business entity that manufactured or caused the manufacture of the sunscreen preparation.

26. The method of claim 13 wherein the step of combining into in a common container swim care articles comprising a disposable swim pant and a sunscreen preparation is performed by the same business entity that manufactured or caused the manufacture of the swim pant.

27. A kit for promoting an array of swim care products, the kit comprising:
a package system that associates the disposable swim pant and the sunscreen preparation of claim 1 for joint sale to a shopper.

28. The kit of claim 27 wherein the package system comprises a tote.

29. The kit of claim 27 wherein the sunscreen preparation is packaged in at least one container of less than about 80 ml of sunscreen preparation.

30. The array of claim 27 wherein the sunscreen preparation is packaged in at least one container of less than about 60 ml of sunscreen preparation.

31. The array of claim 27 wherein the sunscreen preparation is packaged in at least one container of less than about 40 ml of sunscreen preparation.

32. The kit of claim 27 further including information about additional swim care articles selected from the group consisting of: hats, sunglasses, swim wear, foot wear, swim toys, towels, disposable sheets, UV sensors, UV protective clothing, after-sun lotion, and water bottles.

33. The kit of claim 27 wherein the sunscreen preparation is pre-applied to a mitt.

34. The kit of claim 27 wherein the sunscreen preparation is pre-applied to a wipe.
